(19) United States
(12) Patent Application Publication
(10) Pub. No.: US 2008/0108925 A1
(43) Pub. Date: May 8, 2008

(54) WOUND DRESSING

(76) Inventors: Martin C. Kaplan, Rochester, NY (US); Manju Rajeswaran, Fairport, NY (US); Yannick Joseph Lerat, Mellecey (FR); Jean Michel Guilment, Virey Le Grand (FR); Nelson A. Blish, Rochester, NY (US); Andrew F. Kurtz, Macedon, NY (US)

Correspondence Address: Susan L. Parulski, Patent Legal Staff;Carestream Health, Inc., 150 Verona Street, Rochester, NY, 14608, US

(21) Appl. No.: 11/936,812

(22) Filed: Nov. 8, 2007

Related U.S. Application Data
(62) Division of application No. 11/305,856, filed on Dec. 16, 2005.

(51) Int. Cl. A61F 13/02; A61L 15/12

(52) U.S. Cl. 602/50

(57) ABSTRACT

A dressing for wounds includes a useful material, such as dye or biocide, trapped within or behind a gelatin barrier. On application to a wound, metalloproteinases naturally present in the wound diffuse into the dressing and degrade the gelatin, releasing the trapped material. The released material serves various useful purposes, such as indicating the status of the wound or improving wound healing.

### CROSS REFERENCE TO RELATED APPLICATIONS

This is a Divisional of commonly assigned application U.S. Ser. No. 11/305,856 entitled “WOUND DRESSING”, filed on Dec. 16, 2005 in the names of Kaplan et al., and which is assigned to the assignee of this application.

### FIELD OF THE INVENTION

This invention relates to a device and method for dressing wounds, and more particularly to a dressing containing gelatin that degrades on contact with the wound, thereby releasing material that indicates the status of the wound or improves healing of the wound, or both.

### BACKGROUND OF THE INVENTION

Wound healing is a major concern of healthcare providers and governmental organizations worldwide. Costs for wound treatment are enormous. Health and quality of life can be severely affected. Wound care often is labor intensive, requiring frequent attention by skilled professionals. Aging populations will increase the need for wound care.

Current approaches to treatment of wounds include a variety of dressings, often designed to control wetness and humidity, to keep out bacteria, and to apply antimicrobial agents and growth factors. Progress of healing often is monitored by simple techniques such as measuring the diameter of the wound, intrusive probing to determine the depth of the wound, and qualitative visual assessment.

An improved dressing can improve both the rapidity of healing and the quality of the outcome, including reducing infection, pain and scarring. An improved dressing also can reduce cost, even if the dressing is more expensive, by improving the rate of wound healing and thereby reducing the duration of treatment, and by allowing for less frequent and simpler attention by medical professionals, reducing labor costs. Improved methods for monitoring wound healing can facilitate better choice of treatment, and reduce costs by allowing for less frequent attention by medical professionals.

Wound healing is a complex process, which includes the production of a class of chemical compounds known as metalloproteinase (MMP). MMP is a family of proteases present in wounds for the purpose of breaking down damaged tissue for removal from the wound site. The control of inflammation and MMPs is essential, as the proteases not only degrade new tissue but also denature growth factors. Natural tissue inhibitors of MMPs are present, but often do not increase sufficiently to counteract an increase in MMPs. The result can be impaired healing. This is especially problematic in chronic wounds, which can linger for months or even years, despite the application of appropriate treatments. Excess proteinase activity is now considered to be a major cause of impaired healing, by destroying new tissue and growth factors. There is an increased expression of MMPs in a burn wound. MMPs involved in wounds are: collagenase (MMP-1, MMP-8), gelatinases (MMP-2, MMP-9) and elastase (MMP-13).

The analysis of MMP concentration is a good indicator of wound healing status. MMPs are known as biomarkers of wound healing, together with cytokines, and many other biochemical molecules (see Moseley et al. in British Journal of Dermatology, 150, 2004, pp. 401-413. for a review). MMP-2 and MMP-9 are ranked as very good indicators of wound healing by the authors cited above. Both MMPs are gelatinase enzymes.

The level and type of MMPs present in a wound also is symptomatic of bacterial infection. Bacteria produce two primary types of toxins, distinguished by their chemical makeup, their source, and the mechanism of their release from the bacteria: exotoxins and endotoxins. Endotoxins are associated with gram-negative bacterial species only, and are composed of lipopolysaccharides. Endotoxins in the wound environment have been found experimentally to stimulate the production of inflammatory mediators such as TNF-alpha and the interleukins, which in turn induce the production of endogenous matrix metalloproteases (MMPs). Increased levels of MMPs are known to exist in many types of nonhealing wounds and are believed to contribute to the local destruction of growth factors, receptors, and tissue components. Clinical and research data demonstrate that bacterial endotoxins have a detrimental effect on wound tensile strength. Endotoxins have been found to decrease collagen deposition and cross-linking and are associated with surgical wound dehiscence. Therefore, a dressing or method for detecting the level and type of MMPs present in a wound could be indicative of the bacterial load present in that wound.

Currently, MMPs are quantified by laboratory methods requiring sampling of wound fluid and chromatographic analysis. This cannot be used for field analysis, and is too expensive for systematic analysis.

### SUMMARY OF THE INVENTION

Briefly, according to one aspect of the present invention a dressing has a first material that is degradable by a metalloproteinase. A second material is confined by the first material, which is released upon degradation of the first material, which causes a change in the spectral properties of the dressing, and the dressing maintains contact with the wound.

The invention relies on the ability of metalloproteinases (MMPs) to digest proteins, especially gelatin. Its primary object is to release useful material into a wound, or within a wound dressing, or more generally anywhere that MMPs may be present. The useful material variously serves to treat the wound, to diagnose, or to monitor healing progress.

Various wound dressings are disclosed, generally comprising gelatin and a second material. The gelatin acts as a barrier or anchor, preventing the release or diffusion of the second material. Upon application of the dressing to a wound, MMPs naturally present in the wound diffuse into the dressing. These MMPs degrade the gelatin, thereby releasing the second material. Different embodiments use a variety of second materials, including dyes and dye precursors to change the color of the dressing, and healing agents such as growth factors and keratinocytes that diffuse into the wound and promote healing. MMP concentration in the wound is an indicator of the status of the wound, and generally excessive MMP concentrations are present in poorly healing wounds.

The various dressing structures include layers of gelatin mixed with dyes (or dye precursors or healing agents), layers of gelatin embedded with droplets of dye, layers comprising microcapsules of gelatin and dye, and layers of gelatin obstructing other layers containing dye.

The invention and its objects and advantages will become more apparent in the detailed description of the preferred embodiment presented below.

### BRIEF DESCRIPTION OF THE DRAWINGS

FIG. 1A shows a wound dressing having a layer of gelatin containing droplets of dye, and droplets are released upon reaction of the gelatin with MMP from the wound, allowing the dye to diffuse into a mordant layer, thereby changing the color of the mordant layer, which is observable through the transparent backing of the dressing.

FIG. 1B shows a wound dressing having a layer of gelatin containing droplets of dye, and droplets are released upon reaction of the gelatin with MMP from the wound, allowing the dye to diffuse into a mordant layer, thereby changing the color of the mordant layer, which is observable through the transparent backing of the dressing, and a barrier layer to reduce leakage of dressing components into the wound.

FIG. 2 shows a wound dressing component having a layer of gelatin mixed with dye, which is released upon reaction of the gelatin with MMP from the wound, allowing the dye to diffuse into a mordant layer, thereby changing the color of the mordant layer which is observable through the transparent backing of the dressing.

FIG. 3 shows a wound dressing component having two layers of gelatin mixed with two different dye precursors, which are released upon reaction of the gelatin with MMP from the wound, allowing the dye precursors to react with each other, yielding a dye which diffuses into a mordant layer, thereby changing the color of the mordant layer which is observable through the transparent backing of the dressing.

FIG. 4 shows a wound dressing component having a layer of gelatin containing droplets of two different dye precursors, which are released upon reaction of the gelatin with MMP from the wound, allowing the dye precursors to react with each other, yielding a dye which diffuses into a mordant layer, thereby changing the color of the mordant layer which is observable through the transparent backing of the dressing.

FIG. 5 shows a wound dressing component having two layers, one of gelatin and one of collagen. Each layer contains two different dye precursors. Certain MMPs present in the wound will preferentially degrade the gelatin, while other MMPs will preferentially degrade the collagen. Upon such degradation, the dye precursors are released and react with each other, yielding one color dye from the gelatin layer, and a different color dye from the collagen layer. Both dyes diffuse into the mordant layer, altering the mordant layer color. The resulting color indicates the concentration of the two different types of MMP, so that the single dressing allows monitoring that discriminates between different types of MMP.

FIG. 6 shows a wound dressing having a layer containing microcapsules of gelatin mixed with dye. Upon degradation of the gelatin by MMP from the wound, the dye is released and diffuses into the mordant layer, changing the color of the dressing.

FIG. 7 shows a wound dressing having a layer of gelatin. MMPs from the wound diffuse into the dressing and degrade the gelatin, thereby consuming the MMPs. This reduces the concentration of excessive MMPs in a poorly healing wound.

FIG. 8 shows a wound dressing in which a layer of gelatin obscures an underlying layer of dye. MMPs from the wound degrade the gelatin, exposing the dye and thereby changing the color of the dressing.

FIG. 9 shows a wound dressing in which a mordant layer and a dye layer are separated by a barrier layer of gelatin. MMPs from the wound degrade the gelatin barrier layer, allowing dye to diffuse into the mordant layer, thereby changing the color of the dressing.

FIG. 10 shows a wound dressing in which a layer of healing agent, such as growth factor, is separated from the wound by a gelatin barrier layer. MMPs from the wound degrade the gelatin, allowing the healing agent to diffuse into the wound and promote healing.

FIG. 11 is a perspective view showing a wound dressing with multiple strips.

### DETAILED DESCRIPTION OF THE INVENTION

The invention is a wound dressing containing a first material (barrier material), which confines a second material (active material) useful in promoting, diagnosing or monitoring wound healing. The barrier material is composed of gelatin or other material that it is degraded by one or more of the MMPs (metalloproteinases) naturally present in a wound. Degradation of the barrier material releases the active material, which then promotes healing of the wound and/or indicates the status of the wound by the concentration of the released second material. In various embodiments, there may be more than a single barrier material, and more than a single active material.

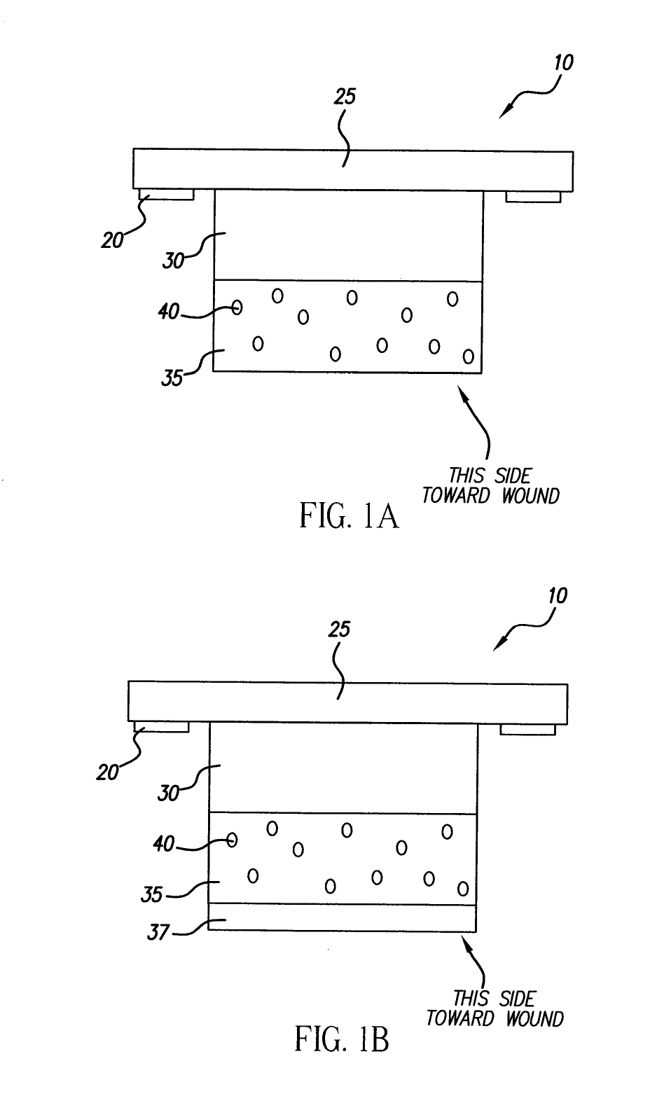

The preferred embodiment for a dressing 10 is shown in FIG. 1A. Droplets 40 of dye in an oil solvent are dispersed within a layer of gelatin 35. A backing 25 of transparent urethane provides mechanical support for the dressing components, and holds the dressing against the wound with the help of adhesive 20 at the edges of the dressing. Upon application of the dressing to a wound, MMPs naturally present in the wound diffuse into the gelatin layer, degrading the gelatin. This releases the dye droplets, which diffuse. Some of the dye diffuses into an opaque, white mordant layer 30, thereby coloring the mordant layer. The colored mordant layer may be observed from above (i.e. from the side of the dressing away from the wound), through the transparent backing 25, by eye or optical instrument (not part of this invention), to assess the color and thereby assess the concentration of MMPs present in the wound. Various optical instruments may be used, including a spectralradiometer, a calorimeter, or a densitometer. Alternatively, the color may simply be observed by eye, possibly assisted by comparison with a calibration chart exhibiting different colors corresponding to different MMP concentrations.

Changes of the dressing color may be observed over a period of time, thereby assessing changes in wound MMP concentration.

Where different parts of the wound are healing differently, the MMP concentration will vary accordingly. The color of the dressing will be non-uniform, representing the MMP concentration in the part of the wound directly below each part of the dressing. Thus, the color of the dressing provides an image of the MMP concentration varying across the wound. This image may be observed by eye, or may be captured by optical imaging devices, such as a camera or imaging spectralradiometer. This information can help in the diagnosis and treatment planning for wounds, revealing which parts of the wound are healing well, and which parts are not.

Although the above preferred embodiment uses droplets of dye in oil solvent, alternative embodiments can use other solvents, or no solvent at all.

As used above, and throughout this description of the invention, the term “mordant” refers to any material that tends to trap and hold dye, preventing or reducing its further diffusion. Commonly, a mordant is a metallic salt that combines chemically with the dye to fix or set the dye permanently. Typically, such a mordant is either inherently collodial or produces colloids, and can be either acidic or basic. Exemplary, generally known, mordants include tannic acid, alum, chrome alum, and certain salts of aluminum, chromium, copper, iron, potassium, and tin. The mordant layer 30 can include one or more mordants, as well as binding, filler, colorants, and structural components. Titanium dioxide particles are a suitable white colorant. (Titanium dioxide is the most common white pigment in white paints.) The white mordant layer 30 serves several purposes. It hides the appearance of the wound, which otherwise might be observable through the dressing, possibly confounding the optical measurement of the dye. The white mordant layer 30 also traps the dye (and may also trap other chemical components such as the oil solvent), preventing it from spreading elsewhere and thereby both enhancing the observable color change (by reducing the loss of dye to other parts of the dressing and the wound), and reducing the bioburden of dye that otherwise may spread to the wound. The white mordant layer 30 also may be designed to encourage the spread of the dye over a [usually small] area of the mordant layer. Spreading the dye decreases the optical density at the original location of the dye, but by covering a larger area increases the total signal (optical density) averaged over the entire dressing. Conversely, if the signal is too strong (such as for a highly absorbing dye), the mordant layer may be designed to limit the spread of the dye, thereby reducing the strength of the color to avoid highly saturated colors that are difficult to measure optically. (It is well known that spreading a certain volume of dye over a larger area increases the total optical absorption by the dye over that larger area, as opposed to the total optical absorption over that same area with the dye concentrated in only part of the area. This is explained in more detail below.)

The white mordant layer may be replaced by two layers, having a white layer below a fully or partially transparent mordant layer. This two-layer structure may provide a stronger optical signature from the diffused dye, because the dye trapped by the transparent mordant layer is entirely above (meaning toward the transparent dressing backing) the white layer, and thus not obscured by the white layer. Conversely, for a single white mordant layer, dye trapped in within the layer is partially obscured by the part of the white mordant layer above the dye. Whether to use a single or dual layer depends on issues such as manufacturing costs and whether the dye signature is sufficiently strong. Throughout this description of the invention, where a mordant layer is mentioned, alternative embodiments are possible and intended, using a dual layer in place of a single layer mordant. Although the mordant layer 30 is white in the preferred embodiment, alternative embodiments may use other colors. It usually is preferred simply that the color of the mordant be different from the color of the dye, to provide observable contrast. It also is possible to use the same color of mordant as color of dye, in which case the diffusion of the dye into the mordant will simply increase the strength (optical density) of the color, which is observable with optical instruments. Similarly, although the preferred embodiment above uses a transparent urethane backing 25, alternative embodiments may use different transparent backings, or partially transparent backings.

The dressing 10 may also be equipped with a barrier layer 37, as shown in FIG. 1B, which can allow moisture vapor to exit the wound, while preventing moisture and bacteria from entering the wound. Barrier layer 37 also allows MMPs to enter the dressing 10 from the underlying wound site. Barrier layer 37 may also be used to minimize the amount of gelatin 35 (or other encapsulating proteins such as collagen) that enters the wound site.

Typically, a wound dressing may include other components, such as hydrogels, alginates, or silver compounds. Such components could be incorporated within the dressing, or between the dressing and the wound. This invention does not require, nor claim, such components, although it may be desirable to include them in the dressing for reasons apart from this invention. Additionally, it should be understood that the wound dressing 10 could have a modular construction or interaction with other dressings. For example, a hydrofiber or foam dressing could be applied within a wound as the primary dressing, with wound dressing 10 then serving as a secondary dressing or bandage which is applied to the surrounding tissue (skin) surface with adhesive 20, such that the inner layer (such as gelatin layer 35 of FIG. 1A) is in contact with the outer surface of the primary dressing. Wound exudates (including MMPs) could be absorbed by the primary dressing, and from there, into dressing 10, with a color change then following with respect to the MMP content. Under appropriate circumstances, a given wound dressing 10 of the present invention could be used as a secondary dressing, such that a first wound dressing 10 is applied and records a color change. It could then be removed, and a second wound dressing 10 could be applied, while the primary dressing may remain intact and undisturbed at the wound site.

As used throughout this patent, the term “dye” refers to any material having a desirable spectral absorption and/or fluorescence, including materials where the spectral absorption and/or fluorescence may be partly or primarily in non-visible portions of the spectrum, such as in the infrared or ultraviolet; and including such aforesaid materials even if commonly referred to by other names, such as pigments.

A suitable dye for the embodiment shown in FIG. 1A is β carotene. It is oil soluble, and non-toxic. (It provides the orange color in carrots.) Another suitable dye is “Solvent Yellow 18,” available from Saujanya Dye Chem, Gujarat, India. Solvent Yellow 18 is another food dye, non-toxic and oil soluble. Many other dyes also are suitable. Many oils are suitable as the solvent in the oil droplets, such as vegetable oils. Non-toxic material are preferable, to minimize harm from any unintended leakage from the dressing into the wound. In an alternative embodiment, the droplets of dye may use a solvent other than oil, or no solvent at all. The droplets may be of any shape, although typical manufacturing processes tend to produce approximately spherical droplets. The droplets need not be liquid; they may be crystalline, amorphous, gaseous, other phases (such as liquid crystals), or a mix of phases.

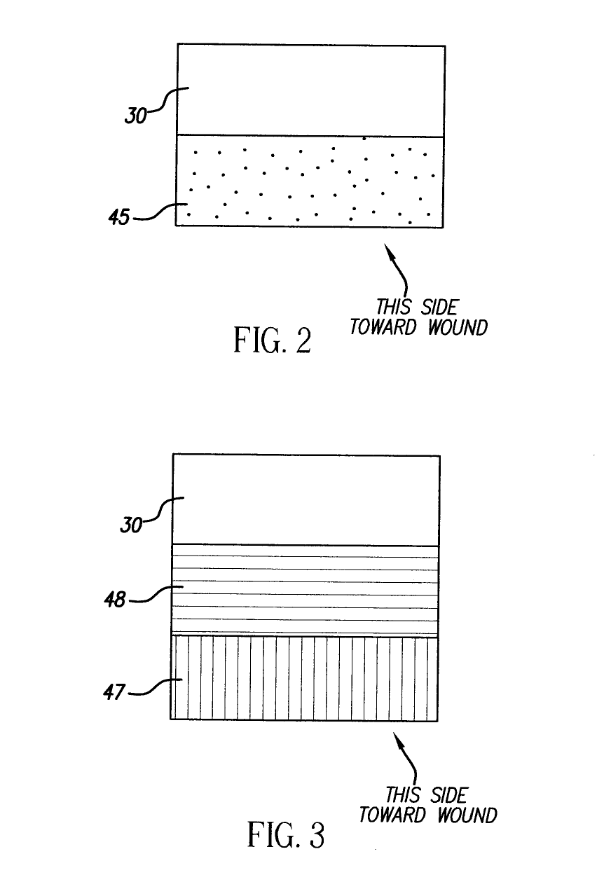

In another embodiment, the dye simply may be mixed with the gelatin, rather than confined to droplets within the gelatin. This is shown in FIG. 2. A layer 45 comprising a mixture of gelatin and dye, and a second layer comprising a white mordant 30 is shown. As in FIG. 1, the dressing also has a transparent backing and adhesive, but for clarity these are not shown in FIG. 2 or some later figures, since they perform the same role as in FIG. 1. Upon application of the dressing to the wound, MMPs naturally occurring in the wound diffuse into the layer of gelatin and dye mixture 45, and degrade the gelatin. Dye previously confined by the degraded gelatin is released, and diffuses in part into the mordant layer 30, coloring the mordant layer. The color may be observed by eye or optical instrument through the transparent backing.

In yet other embodiments, the mordant layer 30 may be transparent rather than opaque, or partially transparent. It may be colored, or clear. With a fully or partially transparent mordant, it is possible that undiffused dye droplets in the gelatin layer may be observable through the mordant layer; thus the dressing would appear to have the color of the dye even before MMPs degrade the gelatin and release the dye. However, degrading of the gelatin and diffusion of the dye still will change the color of the dressing, even though the dye does not change chemically. This is because a dye that is spread over an area absorbs more light than the same volume of dye in compact droplets. This increased absorption from dispersing a dye is a well-known effect. A simple example involves drawing a dark black circular dot on a transparency (such as used with an overhead projector). Light flooding the transparency will be partly absorbed by the dot. Drawing a second, identical black dot atop the first (i.e. adding more ink to the original dot) will have little effect on the amount of light passing through the transparency from below, because the first dot absorbs almost all the light impinging on the dot. The second dot (placed atop the first, in the identical area of the transparency) does absorb some of the small amount light that passes through the first dot, but because this is a small amount of light, it has a small effect, very slightly decreasing the total amount of light passing through the entire transparency. However, if the second dot is placed not atop the first dot, but to the side of the first dot, the second dot will be as effective as the first dot in absorbing light, thus doubling the opacity of the transparency. Another common example is seen in inkjet printing. Great care is taken in the design of inkjet papers, to control the spread of the deposited dyes, because a change in the spreading causes a change in the color of the print. Thus, to control the color of the print, the dye spreading must be controlled.

In the above preferred embodiment, it often is desirable for the mordant layer 30 to be thin. This is because the volume of dye will spread over a larger area for a thin mordant layer than for a thick mordant layer, and covering a larger area will increase the color change, as described above. However, in other embodiments, thicker mordant layers may be used for a variety of reasons, such as improved manufacturability, adequate thickness to make the mordant layer opaque, to reduce the color change if the dye is an excessively strong absorber, or to provide more complete capture of the dye to better prevent diffusion elsewhere.

The mordant layer in the various embodiments serves several useful purposes. It stabilizes the physical position of otherwise diffusing dyes, holding the dyes near the transparent dressing backing where the dyes may be observed without also seeing other dressing components, wound exudate in the dressing, or even the wound itself which may be visible through the dressing. If these other components were seen (by eye or by optical instrument), it would somewhat confuse the signal from dye with the unintended signal from the other components. The mordant also serves as a “sink” for the diffusing dye, trapping it near the dressing backing, thereby increasing the dye concentration near the backing by providing a bias (preferred direction) for the dye diffusion. This diffusion bias also tends to reduce dye diffusion into the wound, thereby reducing the bioburden of foreign material in the wound. The mordant layer typically is white, allowing the dye diffused into the mordant to be observed against a white background to avoid confounding the dye optical signature with other dressing and wound components hidden behind the white mordant. Other colors or mordant, including a clear mordant, also are possible.

However, the mordant is not essential to this invention. Any of the embodiments using a mordant may be modified simply by eliminating the mordant. Without a mordant layer, released dye will tend to diffuse more uniformly throughout the dressing, and generally will not present itself again a white background. Nevertheless, the dye can be optically observed through the transparent backing of the dressing, indicating the MMP concentration in the wound. There are various reasons why it may be desirable to eliminate the mordant layer, including reduced manufacturing cost, improved physical properties of the dressing such as flexibility, and low dye mobility insufficient to effectively diffuse across the dressing into the mordant, rendering the mordant largely useless.

Alternatively, in the many embodiments using a mordant layer 30, the mordant layer may be placed below, rather than above, the gelatin layer. This tends to decrease the observable signal (depending on the transparency of the gelatin and other materials that over the mordant layer), but has advantages, such as better preventing the dyes and solvents, released from the gelatin layer, from diffusing into the wound, because to reach the wound they must diffuse through the mordant layer, which is chemically designed to trap them. In this case, mordant layer 30 may serve at least some of the functions previously described for barrier layer 37. Preventing diffusion of these materials into the wound is desirable to minimize the bioburden.

FIG. 3 shows another alternative embodiment. A first layer 47 contains a mixture of gelatin and a first dye precursor. A second layer 48 contains a mixture of gelatin and a second dye precursor. Above these layers is a white mordant layer 30. After the dressing is applied to a wound, MMPs naturally present in the wound diffuse into the dressing and degrade the gelatin, releasing the first and second dye precursors. Some of the released first dye precursor reacts with some of the released second dye precursor, chemically reacting to form a dye. Some of this dye diffuses into the white mordant layer 30, thereby changing the color of the dressing, which color change may be observed by eye or optical instrument. As used herein, the term “dye precursor” means any material that, upon reacting with another material, changes color. As above, a change in color generally means a change in the spectral absorption and/or fluorescence, not necessarily in the visible part of the spectrum. In this embodiment, the two different dye precursors react with each other to create a color change. In an alternative embodiment, only a single layer of gelatin is used, containing the first dye precursor. The second dye precursor is elsewhere in the dressing, such as in the mordant layer. The terminology “color change” includes a decrease in color, and thus, for example, a bleaching agent may be used as one of the dye precursors.

In all the above embodiments, as well as embodiments below, the layering morphology for the gelatin is convenient for manufacturing, but not essential to the operation of the invention. Thus, other embodiments may use gelatin in different morphologies, such as spheres of gelatin mixed with dye or dye precursor, or patches of gelatin, blobs of gelatin, or any other shape. Similarly, the white mordant need not be layered, although a layer does have advantages such as manufacturability, uniform sensitivity across the area of the dressing, and good visibility.

For the various embodiments described above that use dye in gelatin (including both dye mixed in gelatin and droplets of dye dispersed in gelatin), the dye may be replaced by a dye precursor, resulting in still more embodiments.

Additional embodiments use dye mixed in gelatin together with droplets dispersed in gelatin, in effect combining some of the above embodiments into alternative embodiments. For example, for the embodiment shown in FIG. 3, the gelatin/dye mixture layer 45 may be replaced by a layer of gelatin containing dispersed dye droplets.

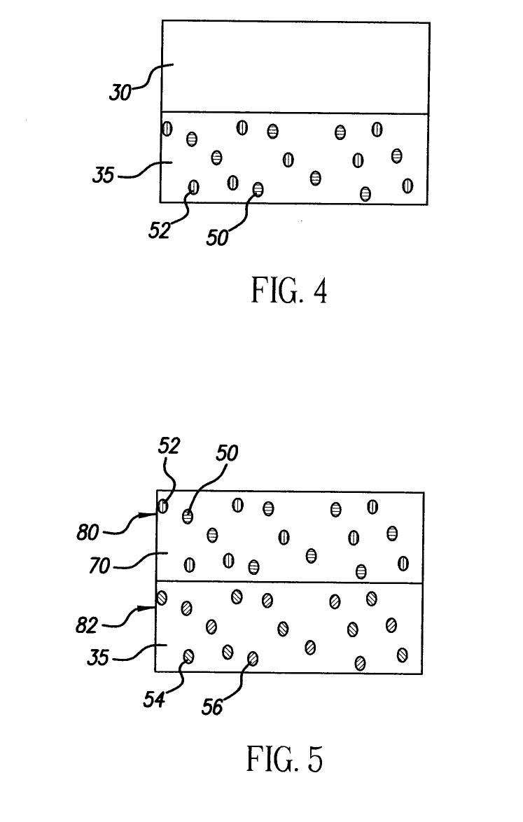

Another embodiment is shown in FIG. 4. A mordant layer 30 is above a gelatin layer 35. Droplets containing a first dye precursor 50 (which may or may not include solvent) are dispersed within the gelatin layer 35. Droplets containing a second dye precursor 52 (which may or may not include solvent) are dispersed also within the same gelatin layer 35. When MMPs diffuse from the wound into the gelatin layer, and degrade the gelatin, the droplets are released and diffuse. Some droplets containing the first precursor encounter some droplets containing the second precursor, and the two precursors react, producing a dye. The dye diffuses into the mordant layer, producing an observable change in the color of the dressing.

A typical color photographic emulsion, of the type known as “incorporated coupler,” is a common example of dye precursor droplets, typically comprising dye precursor chemicals in oil solvent, dispersed throughout a gelatin. Further, a color photographic emulsion typically has layers of gelatin with different dye precursor droplets in different layers. Although photographic emulsions often have only a single dye precursor in each layer, it is straightforward to mix droplets of different dye precursors within any of the gelatin layers. In photographic emulsions, this mixing of different precursors within a single layer is not generally done, because the dye precursors are not intended to react with each other, but rather a “developer” chemical is introduced during photofinishing, which reacts with silver halide also present in the emulsion, resulting in a “redox” by-product, which in turn reacts with the dye precursor (termed a “coupler” in photographic science). Photographic film manufacturing techniques are the preferred, but not only, method for manufacturing the single- and multi-layered gelatin with dye precursor dressing components described above. These manufacturing methods also may be used for the preferred embodiment above, which uses dyes rather than dye precursors, simply by substituting dyes in place of dye precursors in the manufacturing process.

Wounds naturally produce various different MMPs, which target different proteins. A few MMPs mentioned earlier are: collagenases (MMP-1, MMP-8), gelatinases (MMP-2, MMP-9) and elastase (MMP-13). By choosing a particular gelatin for the dressing, or instead choosing another protein degraded by MMPs, such as collagen, the dressing will be preferentially sensitive to a particular MMP (or to several MMPs which all may target the protein). Thus, the dressing can be made to distinguish between different MMPs that may be present in the wound. Alternative embodiments to all the above embodiments use such proteins instead of gelatin. FIG. 5 shows another embodiment having two layers with four dye precursors. (A white mordant may, or may not, be included.) A first layer 80 comprises a collagen 70 degradable by MMP-1, and droplets of a first dye precursor 50 and a second dye precursor 52. A second layer 82 comprises a gelatin 35 degradable by MMP-9, and droplets of a third dye precursor 54 and a fourth dye precursor 56. Upon application of the dressing to the wound, MMPs present in the wound diffuse into the dressing. Any MMP-1 present preferentially degrades the collagen 70 in layer 80, releasing precursors 50 and 52, which react with each other to form a dye of a specific color, e.g. a red dye. Any MMP-9 present preferentially degrades the gelatin 35 in layer 82, releasing precursors 54 and 56, which react with each other to form a dye of a different specific color, e.g. a blue dye. Thus, the dressing is able to distinguish between MMP-1 (by the resulting redness of the dressing) and MMP-9 (by the resulting blueness of the dressing). An optical device such as a spectralradiometer (not part of the invention) may be used to measure the spectral absorbing of the dressing, and thereby determine quantitatively the concentration present of MMP-1 and of MMP-9. The precursors from different layers may react (such as precursor 52 reacting with precursor 56), and the compound resulting from the reaction may be colorless or colored, and thereby may either have no effect on the color of the dressing, or may further alter the color of the dressing to provide improved sensitivity and ability to distinguish between MMP-1 and MMP-9 concentrations.

Obvious alternative embodiments use different proteins in place of the particular collagen and gelatin, different dye precursors possibly resulting in different color dyes, and also may use more than two layers to distinguish more than two MMPs. Also, additional layers may be used to improve the dynamic range, for example with one layer responding to low concentrations of MMP-9, but the color of the layer saturating at moderate MMP-9 concentrations, and a second layer responding more weakly (i.e. with a less colorful dye) and thereby not saturating until higher concentrations of MMP-9 are present. Obviously, the two different layers must use different colored dyes, so that the saturation of the first layer does not obscure the color signal of the second layer. Additional embodiments may use mixtures of proteins with each layer, providing sensitivity to more MMPs without the complexity of still more layers and more dye precursors. Other alternative embodiments use dyes, rather than dye precursors, as described earlier for gelatin-based embodiments.

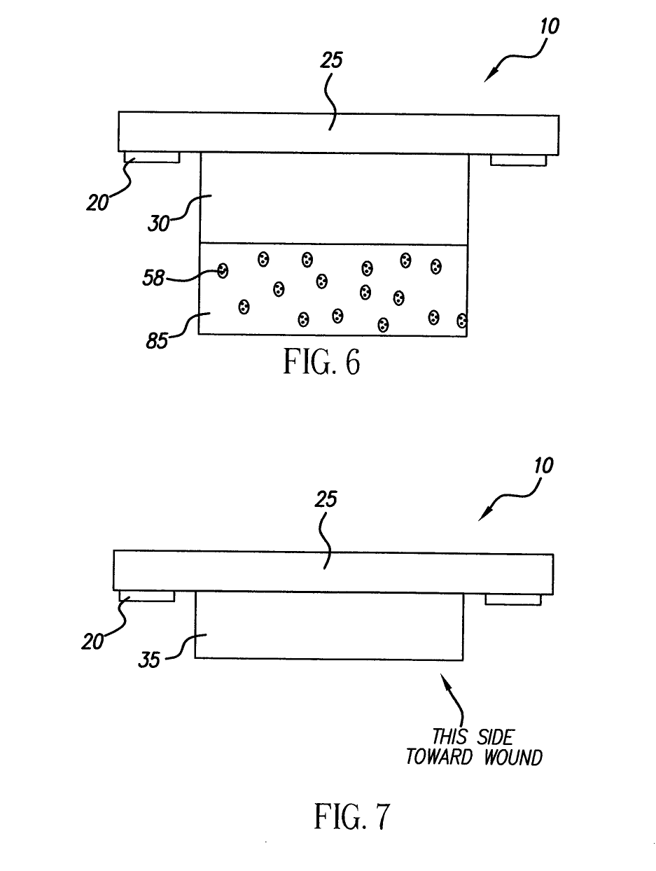

Additional alternative embodiments use microencapsulation to confine the dyes or dye precursors within microcapsules of gelatin. One such embodiment for dressing 10 is shown in FIG. 6. A porous layer 85 contains microcapsules 58. The microcapsules are each a mixture of gelatin and dye. Upon application of the dressing to a wound, MMPs present in the wound diffuse into the dressing, degrading the gelatin in the microcapsules, thereby releasing dye from the microcapsules. Some of the dye diffuses to the mordant layer 30, changing the color of the mordant layer. The change in color may be observed and measured by eye or optical instruments through the transparent backing 25. The transparent backing 25 provides mechanical support for the dressing structures. The adhesive 20 adheres the edges of the backing to the wound, or to the skin adjacent the wound. In this embodiment, the microcapsules are of the type known as monolithic, meaning that they of are a uniform mix throughout each microcapsule. This is in contrast to, for example, mononuclear microcapsules, in which the dye is inside a shell of gelatin. The microcapsules may be manufactured by the coacervation process, which was the earliest microencapsulation process studied, and remains commonly used. One such coacervation process begins with an aqueous solution of gelatin and a water-soluble dye such as the sodium salt of Sunset Yellow (also known as FD&C Yellow No. 6 Lake), available from Saujanya Dye Chem, Gujarat, India. Ethanol is slowly added to the aqueous solution, leading to phase separation and the gradual desolvation of gelatin, together with some of the dye, into coacervates, which are small particles. A more detailed description of coacervation of gelatin into microcapsules is available in the book Microspheres, Microcapsules & Liposomes, MML Series Volume 1, Preparation & Chemical Applications, Reza Arshady, Editor, copyright 1999 by Citus Books, ISBN 0 9532187 1 6, Part Three, Chapter 10, pp. 305-308.

The coacervation process described above is just one of many possible coacervation processes available for preparing monolithic microcapsules of gelatin and dye. Different dyes may be used, and the ethanol may be replaced by other coacervation agents, as described in Chapter 10 of the above-referenced book.

Microencapsulation processes other than coacervation also are available, as described by the above-referenced book. These include the polymerization process (page 290 ff of the book), polycondensation (page 292 ff), solvent extraction and evaporation (page 296 ff), suspension crosslinking (page 302 ff), extrusion (page 312 ff), spraying (page 314 ff), and coating (page 315 ff). The various microencapsulation processes result in various microcapsules, shown in the book on page 282, FIG. 1. These include the above-mentioned monolithic type, mononuclear (dye confined inside a gelatin shell), and various other types of microcapsules each having one or more shells of gelatin enclosing dye. Various embodiments similar to FIG. 6 are possible using the various microencapsulation processes resulting in various microcapsule morphologies. Further, a layer such as layer 85 of FIG. 6, comprising a porous material with microcapsules (not necessarily monolithic) of gelatin (or other protein) and dye (or dye precursor) may be substituted in earlier embodiments in place of layers comprising gelatin and droplets.

The above dressing embodiments allow monitoring the status of a wound by changing the color of the dressing in response to the MMP concentration in the wound. In alternative embodiments now described, the dressing promotes healing of the wound, rather than monitoring the status of the healing. This is achieved by replacing the dye (or dye precursor) by an agent that promotes healing. Upon application of the dressing to the wound, MMP present in the wound diffuses into the dressing, degrading the gelatin (or other protein), releasing the healing agent. Some of the healing agent then diffuses into the wound, where it promotes healing. The concentration of MMP, and consequently the concentration of healing agent released into the wound, varies depending on the healing status of the wound. For these embodiments using healing agents, the backing of the dressing need not be transparent.

One such healing agent is an antimicrobial agent, such as penicillin. Upon degradation of the gelatin by MMP, the antimicrobial agent is released and diffuses into the wound, where its biocidal activity reduces the biocontamination of the wound. The more biocontamination present in the wound, the more resulting dead tissue. This leads to the production of more MMP by the healing process, which increases the degradation of gelatin in the invention, and consequently more antimicrobial agent is released. Thus, the invention has the desirable property of delivering more antimicrobial agent where biocontamination is worse. Many antimicrobial agents may be used, such as Penicillin G and V, 4× Meracilina—4480990 (available from ASTA Medica GmbH, Frankfurt, Germany), and Streptomycin Sulfate (available from Pfizer Inc. New York, N.Y.).

Another such healing agent is an inhibitor (or sequestrant) of MMP activity, such as silver. This provides negative feedback control of the level of MMP activity. When MMP concentration and activity increases, more gelatin is degraded in the dressing, releasing more MMP inhibitor (or sequestrant), reducing the MMP activity. The invention thereby promotes wound healing, by limiting excess tissue destruction by excess MMP activity.

Another such healing agent is a chemical growth factor, such as platelet derived growth factor (PDGF), tumor necrosis factor (TNF), fibroblast growth factor (FGF), transforming growth factor alpha (TGF α), transforming growth factor beta (TGF β), keratinocyte growth factor (KGF), Becaplermin (rhPDGF-BB), epidermal growth factor (EGF), and platelet derived endothelial cell growth factor (PDECGF). Released growth factors can diffuse into the wound bed, where they inhibit excess MMP activity and stimulate tissue repair. The invention controls the release of growth factor, releasing more growth factor where more MMP activity is present. This is desirable both to limit excess MMP activity, and because more tissue damage will lead to more MMP activity, releasing more growth factor to increase repair of the more damaged tissue. Growth factors are available from various vendors, such as Epidermal Growth Factor available from Sigma-Aldrich, CAS Number 62229-50-9, EG/EC Number 2634687, MDL number MFCD01634807, and Platelet-Derived Growth Factor available from BD Biosciences, catalog number 354051.

Another such healing agent is keratinocytes, or fibroblasts. Keratinocytes may be prepared by the method disclosed in U.S. Pat. No. 6,197,330 (Rees et al.) Fibroblasts are available from vendors such as Cambrex, part code CC-2509.

In yet another alternative embodiment of the invention shown in FIG. 7, gelatin 35 is present in the dressing 10. The dressing need not contain any dyes, dye precursors, or any of the previously mentioned healing agents. Upon application of the dressing to a wound, MMPs present in the wound diffuse into the dressing and react with the gelatin, degrading it. These MMPs are themselves consumed by the reaction with the gelatin. Thus, the gelatin acts to destroy MMP, reducing excessive levels of MMP present in the wound, much as described above for MMP inhibitors as healing agents.

In yet other embodiments, the gelatin dye mixtures (such as layer 45 in FIG. 2, or microcapsules 58 in FIG. 6) are replaced by gelatin molecules having chromophores. The chromophore imparts a color to the gelatin molecules. Upon application of the dressing to the wound, MMPs present in the wound diffuse into the dressing and degrade the gelatin. The components of the degraded gelatin, smaller than the original gelatin molecule, diffuse more rapidly. Any diffusing components that contain a chromophore will be colored, in effect acting as a dye, producing a color change in the dressing. Additionally, the color of the chromophore can be affected by the structure of the molecule (gelatin or fragment of degraded gelatin), and thus the MMP induced degradation of the gelatin may cause a color change of the chromophore. The color changes caused both by diffusion of the colored gelatin fragments, and by change of the chromophore color, may be monitored by eye or optical instrument.

In the above embodiments, the chromophore attached to the gelatin may be replaced by any of the healing agents mentioned above, such as antimicrobial agents or MMP inhibitors. In these additional embodiments, MMP induced degradation of the gelatin results in gelatin fragments, some of which have the healing agent attached and diffuse into the wound, where the healing agent promotes healing.

In another alternative embodiment, the gelatin layer or microcapsule confines an inhibitor of MMPs that cannot diffuse inside the wound bed. Reaction of MMP with the gelatin exposes the inhibitor, which then can inhibit additional excess MMP. Such inhibitor compounds can be non-diffusible Ag species for instance, or Zn chelators (see, for example, U.S. Pat. No. 6,599,523 (Cohen et al.)). This has the advantage that the inhibitor does not enter the wound, reducing the possibility of harmful side effects from the inhibitor.

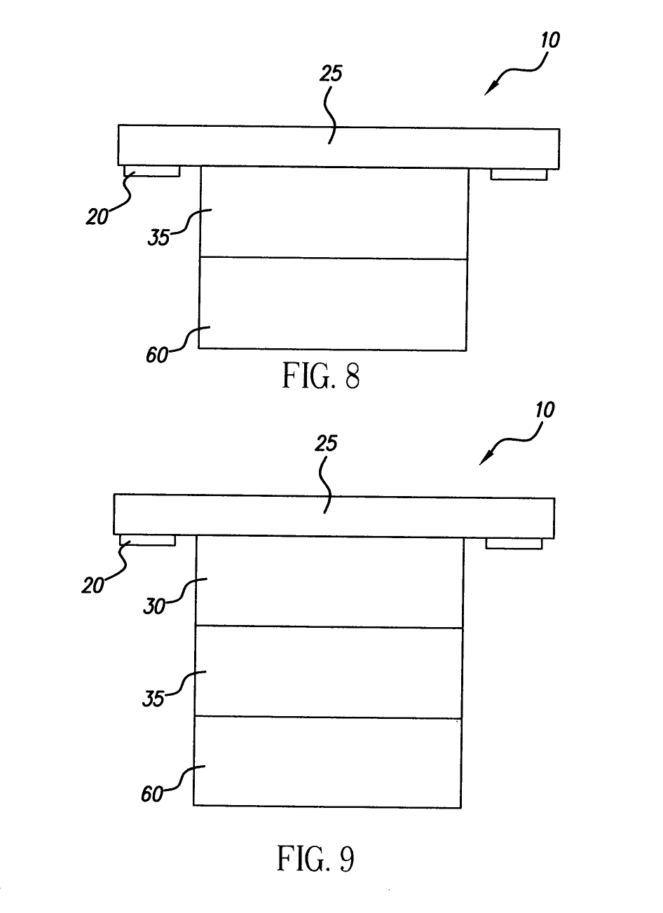

Yet another embodiment is shown in FIG. 8. A layer of dye 60 is below a layer of gelatin 35 in dressing 10. The gelatin is opaque, or colored differently than the dye. Upon application of the dressing to the wound, MMP diffuses into the dressing, degrading the gelatin. Degradation of the gelatin exposes the dye 60, allowing it to be seen through the transparent backing 25. The exposed dye may be monitored by eye or by an optical instrument, looking through the transparent backing.

Another embodiment is shown in FIG. 9. Dressing 10 has a layer of dye 60 below a layer of gelatin 35, and a mordant layer 30 above the gelatin layer 35. Upon application of the dressing to the wound, MMP diffuses into the dressing, degrading the gelatin. This allows dye from layer 60 to leak through the damaged gelatin layer 35, into the mordant layer 30. This changes the color of the mordant layer. The color change may be observed through the transparent backing 25.

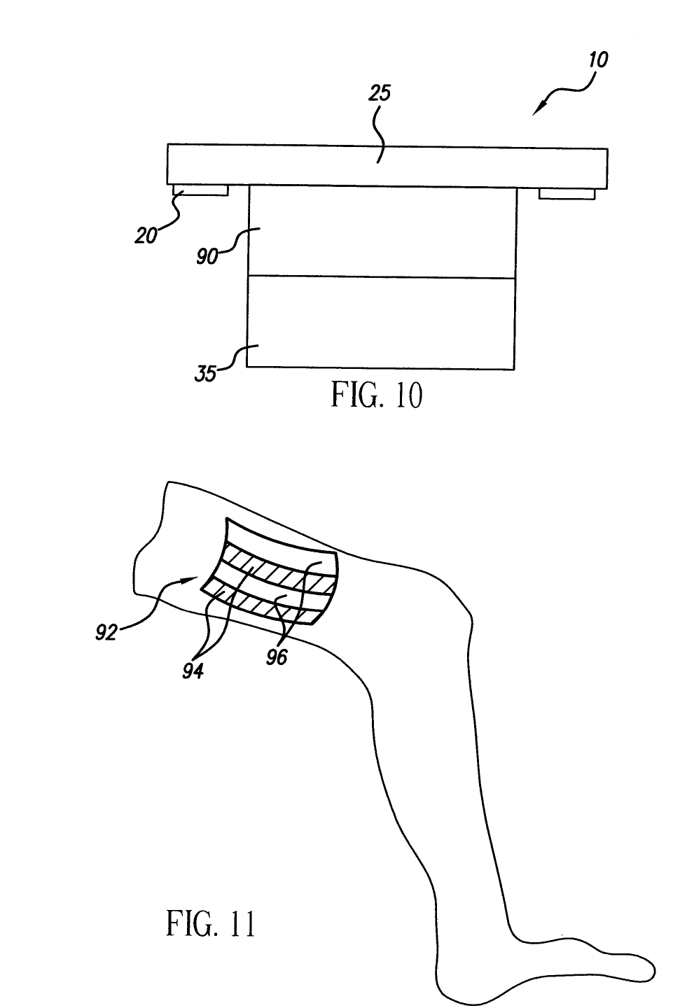

Another embodiment is shown in FIG. 10. Dressing 10 has a layer of healing agent 90 separated from the wound by a layer of gelatin 35. The healing agent may be any of those described above, such as a microbial agent or an MMP inhibitor. Upon application of the dressing to the wound, MMP diffuses into the dressing, degrading the gelatin. Healing agent 90 leaks through the damaged gelatin layer 35, and diffuses into the wound, promoting healing of the wound.

Two references are now mentioned: U.S. Pat. No. 6,713,083 (McGregor et al.) and U.S. Patent Application Publication No. 2004/0044299 (Utsugi).

U.S. Pat. No. 6,713,083 (McGregor et al.) discloses a method for fabricating coated beads, having porous cores and non-porous outer shells. Materials used for the cores and shells include various polymers such as gelatin and collagen, and therapeutic agents such as growth factors and antiseptics. The beads are to be used for implantation into wounds, or wound dressings. U.S. Pat. No. 6,713,083 is distinguishable from the present patent. First, many of the embodiments of the present invention use dyes or dye precursors, for the purpose of revealing the healing status of the wound. U.S. Pat. No. 6,713,083 is limited to therapeutic agents, not using dyes or dye precursors; and U.S. Pat. No. 6,713,083 does not provide nor intend any means to reveal the status of the wound. Second, although some of the embodiments in the present invention use mononuclear microcapsules, which are a form of coated beads, many of the embodiments use uncoated beads, such as monolithic microcapsules, or droplets rather than microcapsules. U.S. Pat. No. 6,713,083 is limited to coated beads. Third, many of the embodiments of the present invention do not use beads at all, such as the embodiment shown above in FIG. 2. Fourth, U.S. Pat. No. 6,713,083 describes coated beads to be used in a dressing, but does not disclose any particular dressing structures in which the beads may be used. Rather, U.S. Pat. No. 6,713,083 is about the beads and the method for manufacturing them, not about how the beads are used within a dressing. Fifth, U.S. Pat. No. 6,713,083 describes one particular method for manufacturing coated beads. There are many other methods for fabricating microcapsules explained in the above-cited reference book Microspheres, Microcapsules & Liposomes.

U.S. Patent Application Publication No. 2004/0044299 (Utsugi) discloses a wound dressing which changes color if there is growth of bacteria in the wound. This is distinguishable from the present invention. First, U.S. Patent Application Publication No. 2004/0044299 monitors bacteria in the wound, while the present invention monitors MMP in the wound (as well as certain embodiments which promote healing). Both bacterial concentration and MMP concentration are important, but for different reasons. Bacterial growth is one of many reasons that wound healing may be impaired. A high MMP concentration sometimes indicates impaired wound healing for non-bacterial reasons. Second, the dressing structures in U.S. Patent Application Publication No. 2004/0044299 are quite different from the dressing structures of the present invention. Third, U.S. Patent Application Publication No. 2004/0044299 mechanism for color change is quite different from the present invention. The present invention relies on MMPs degrading gelatin. Further, in most embodiments in the present invention, the gelatin serves as a barrier constraining a second material (dye or dye precursor), which is released upon gelatin degradation. This is quite different from U.S. Patent Application Publication No. 2004/0044299, which relies on bacterial byproducts reacting with a chemical in the dressing, and thereby causing a change directly in the color of that chemical, rather than by chemically damaging a barrier material and thereby releasing a second material that causes the color change.

The method of MMP detection in a wound or other tissues, by providing dyes or dye pre-cursors within an MMP degradable protein (such as gelatin or collagen), need not be restricted to application by means of a bandage or dressing. For example, the dye and protein combination could potentially be delivered to the wound with a spray. Alternately, the clinician could remove a pre-existing dressing or bandage from the wound site, where the dressing may be coated with, or have absorbed, wound exudates bearing MMPs. The clinician could then spray the MMP degradable proteins bearing dyes (or dye-precursors) onto the removed wound dressing. In either case, a clinician could monitor the wound for color change relatively quickly, compared to waiting for the MMPs to diffuse through a dressing. This could enable a quick assessment of the MMP loading before starting a course of treatment. It should be understood that other forms of dye and encapsulate delivery, aside from a spray (such as a cream or ointment) could be used for the above purpose.

A further embodiment, as shown in FIG. 11, is a wound dressing 92 that comprises a plurality of first strips 94. The first strips 94 have a first material degradable by a metalloproteinase; a second material confined by the first material and released upon degradation of the first material; the release of the second material causes a change in spectral properties of the dressing. A plurality of second strips 96 contains the first material degradable by the metalloproteinase and a third material confined by the first material and released upon degradation of the first material; the release of the third to material releases a healing agent. A means for maintaining the dressing in contact with a wound, for example, adhesive, is provided.

Yet another embodiment consists of a method for determining the burden of metalloproteinases in biological tissues by applying a diagnostic aid comprising a first and second layer. The first layer is in contact with the biological tissue. The second layer has first material, which has a chemical composition that is degradable by a metalloproteinase, and a second material which is confined by the first material and which exhibits changes in spectral properties. The second layer is removed from the first layer after an appropriate time for the metalloproteinases to degrade the first material, while leaving the first layer in contact with the biological tissue. Spectral changes occurring with the second material are observed, and a burden of metalloproteinases, relative to the quantity, type, and location of the metalloproteinases within the tissues is assessed.

A further embodiment consists of a method for determining the burden of metalloproteinases in biological tissues by applying a diagnostic aid which has a first material, which has a chemical composition degradable by a metalloproteinase, and a second material which is confined by the first material and which experiences changes in spectral properties. After an appropriate time for the metalloproteinases to degrade the first material, the diagnostic aid is removed and spectral changes occurring with the second material are observed and a burden of the metalloproteinases, relative to the quantity, type, and location of the metalloproteinases within the tissues is assessed.

The enumeration above of certain embodiments is not meant to exclude other embodiments, such as combinations of the above embodiments.

### PARTS LIST

10 dressing
20 adhesive
25 backing
30 mordant layer
35 gelatin
37 barrier layer
40 dye droplet
45 gelatin and dye mixture
47 layer of gelatin and dye precursor #1 mixture
48 layer of gelatin and dye precursor #2 mixture
50 dye precursor #1
52 dye precursor #2
54 dye precursor #3
56 dye precursor #4
58 gelatin/dye microcapsule
60 dye
70 collagen
80 layer 1
82 layer 2
85 porous layer
90 healing agent
92 wound dressing
94 first strips
96 second strips

1. A dressing comprising:
a first material comprised of a chemical composition that is preferentially degradable by certain metalloproteinases;
a second material confined by the first material and released upon degradation of the first material;
a third material, comprising a chemical composition that is preferentially degradable by certain metalloproteinases, at least some of which are different from the metalloproteinases that degrade the first material;
a fourth material confined by the third material and released upon degradation of the first material, causing a change in the spectral properties of the dressing, such that for different amounts of degradation of the first and third materials, the resulting change in spectral properties of the dressing is different; and
a means for maintaining the dressing in contact with a wound.

2. A dressing comprising:
a layer, parallel to a surface of the dressing away from a wound and spanning most of the area of the dressing, the layer comprising a material which is of chemical composition that is degradable by a metalloproteinase; and
a means for maintaining the dressing in contact with a wound.

3. A dressing comprising:
a first material comprising a chemical composition that is degradable by a metalloproteinase;
a second material confined by the first material and released upon degradation of the first material, the second material comprising an agent that promotes wound healing, wherein either the second and first materials are mixed uniformly throughout a layer, or the second material is in droplets dispersed within a layer of the first material, or the second and first materials are mixed together and the mixture is formed into monolithic microcapsules, or the second material is in a layer farther from the wound than a layer of the first material; and
a means for maintaining the dressing in contact with a wound.

4. The dressing in claim 3 wherein the first material is selected from a group comprising: gelatin, collagen, and elastase.

5. The dressing in claim 3 wherein the first and second materials are mixed together.

6. The dressing in claim 3 wherein the second material is contained in droplets, which in turn are contained within the first material.

7. The dressing in claim 3 wherein the second material is contained inside shells comprising the first material.

8. The dressing in claim 3 wherein the mixture of first and second materials is formed into monolithic microcapsules.

9. The dressing in claim 3 wherein the means for maintaining contact with the wound is a backing with adhesive on the wound-side of the backing near the edges of the backing.

10. The dressing in claim 3 wherein the second material is selected from a group comprising: antimicrobial agent, an inhibitor of MMP activity, silver, a chemical growth factor, keratinocytes, or fibroblasts.

11. A wound dressing comprising:
a plurality of first strips comprising:
a first material degradable by a metalloproteinase;
a second material confined by the first material and released upon degradation of the first material, wherein release of the second material causes a change in spectral properties of the dressing;
a plurality of second strips comprising:
the first material degradable by the metalloproteinase;
a third material confined by the first material and released upon degradation of the first material, wherein release of the third to material releases a healing agent; and
a means for maintaining the dressing in contact with a wound.
